(19) United States
(12) Patent Application Publication
(10) Pub. No.: US 2026/0337517 A1
(43) Pub. Date: Oct. 29, 2026

(54) FRAGRANCE COMPOSITIONS CONTAINING 2,3-BUTANEDIOL AND/OR 1,3-BUTANEDIOL

(71) Applicant: FIRMENICH SA, Satigny (CH)

(72) Inventors: Anne-Victoire DE BAYNAST, Versailles (FR); Madelyne PHAM, Joinville-e-Pont (FR); Aude DAUGERON JOUAULT, LEVALLOIS-PERRET (FR); Addi FADEL, Metuchen, NJ (US); Denis SHCHERBAKOV, Ewing, NJ (US)

(73) Assignee: FIRMENICH SA, Satigny (CH)

(21) Appl. No.: 19/104,893

(22) PCT Filed: Aug. 17, 2023
(86) PCT No.: PCT/EP2023/072634

§ 371 Date: Feb. 19, 2025

Related U.S. Application Data
(60) Provisional application No. 63/374,022, filed on Aug. 31, 2022.

(30) Foreign Application Priority Data
Sep. 30, 2022 (EP) 22198953.6

(51) Int. Cl. C11B 9/00

(52) U.S. Cl. CPC C11B 9/0015; C11B 9/003; C11B 9/0053; C11B 9/0061

(57) ABSTRACT

The present disclosure relates to fragrance compositions, particularly fragrance compositions comprising 2,3-butanediol and/or 1,3-butanediol. The present disclosure also relates to consumer products, such as leave-on products, for instance eau de toilette, eau de parfum, body sprays, deodorants, and the like, containing the said fragrance compositions.

### CROSS REFERENCE TO RELATED APPLICATIONS

This application claims the benefit of priority of U.S. Provisional Application No. 63/374,022, filed Aug. 31, 2022, and European Application No. 22198953.6, filed Sep. 30, 2022, both of which are hereby incorporated by reference as though set forth herein in their entireties.

### FIELD OF THE DISCLOSURE

The present disclosure relates to fragrance compositions, particularly fragrance compositions comprising 2,3-butanediol and/or 1,3-butanediol. The present disclosure also relates to consumer products, such as leave-on products, for instance eau de toilette, eau de parfum, body sprays, deodorants, and the like, containing the said fragrance compositions.

### BACKGROUND OF THE DISCLOSURE

Increasing the solubility of fragrance or fragrance ingredients is a daily challenge in perfumery and cosmetic industries. Ethanol and dipropylene glycol are common solvents for dilution of fragrance oils and concentrates.

With respect to ethanol in particular, controversy exists around the use of ethanol in consumer products, typically personal care products. Such controversy has arisen due to religious conviction, environmental impact, as well as a tendency towards skin irritation. Thus, a growing movement towards decreasing the use of ethanol in personal care products has been observed during the past decade. Many industries, particularly the fragrance industry, are facing a potentially new CARB regulation that seeks to limit the percentage of volatile organic compounds (VOCs) at 50% in personal fragrance products comprising ≤10% fragrance by January 2031.

However, there are challenges to reducing ethanol content in a fragrance product. One challenge is reduction of olfactive impact, which is the efficacy or intensity of a perfumery raw material during the first moments of product performance. As it is very volatile, ethanol aids in providing olfactive impact. Replacing ethanol with a solvent of lower volatility in fragrance products tends to reduce its olfactive impact, which is detrimental since impact is a very important characteristic of a fragrance as it provides the first impression about the fragrance. Another challenge is reduction of solubility of the components of a fragrance. Ethanol is useful in solubilizing fragrance components, many of which are lipophilic. Reduction of ethanol leads to reduction of solubility of fragrance components, often leading to undesirable outcomes. These challenges are more apparent as demands for water-based solutions from consumers are increasing. The technical problem of solubilizing high dosages of fragrances to obtain aesthetically pleasing transparent products without the need for any surfactant or solubilizer becomes another challenge.

Accordingly, there is an ongoing need for fragrance compositions that are free of ethanol but can elicit desirable olfactive performance, provide desirable solubility of fragrance components and/or be aesthetically pleasing, i.e, transparent.

### SUMMARY OF THE DISCLOSURE

The following aspects of the present disclosure seek to address one or more of the problems described hereinabove.

In a first aspect, the present disclosure relates to a fragrance composition comprising at least:
a) 2,3-butanediol and/or 1,3-butanediol, and
b) a fragrance component,
wherein the fragrance composition is free of ethanol and is a homogeneous and transparent solution.

In a second aspect, the present disclosure relates to a consumer product comprising the fragrance composition described herein.

In a third aspect, the present disclosure relates to a method for enhancing or modulating the perceived olfactive impact and/or long lastingness of a fragrance composition, the method comprising adding 2,3-butanediol and/or 1,3-butanediol to a fragrance component, in the absence of ethanol, to obtain the fragrance composition as a homogeneous and transparent solution.

### BRIEF DESCRIPTION OF THE FIGURES

FIG. 1 shows a comparison of the olfactive performance between a reference composition and an inventive composition, both of which contain the same fragrance.

FIG. 2 shows the evaporation rate of the fragrance component of comparative and inventive formulations according to the present disclosure.

FIG. 3 shows the evaporation rate of the solvent component of comparative and inventive formulations according to the present disclosure.

### DETAILED DESCRIPTION

As used herein, the terms “a”, “an”, or “the” means “one or more” or “at least one” unless otherwise stated.

While compositions and methods are described in terms of “comprising,” “containing,” or “including” various components or steps, the compositions and methods can also “consist essentially of” or “consist of” the various components, substances and steps. As used herein the term “consisting essentially of” shall be construed to mean including the listed components, substances or steps and such additional components, substances or steps which do not materially affect the basic and novel properties of the composition or method. In some embodiments, a composition in accordance with embodiments of the present disclosure that “consists essentially of” the recited components or substances does not include any additional components or substances that alter the basic and novel properties of the composition.

Unless defined otherwise, all technical and scientific terms used herein have the same meaning as is commonly understood by one of skill in the art to which this specification pertains.

It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, and unless otherwise indicated, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Throughout the present disclosure, various publications may be incorporated by reference. Should the meaning of any language in such publications incorporated by reference conflict with the meaning of the language of the present disclosure, the meaning of the language of the present disclosure shall take precedence, unless otherwise indicated.

Throughout the present disclosure, various chemical names and structures may be recited. Unless otherwise stated, any stereoisomers, such as enantiomers, diastereomers, anomers, epimers, and the like; and geometric isomers, such as cis/trans or E/Z isomers, of the recited chemical name or structure are contemplated. As would be understood by those of ordinary skill in the art, stereoisomers may possess one stereocenter, giving rise to enantiomers, or more than one stereocenter, giving rise to diastereomers, each stereocenter having one of two different stereochemistries (i.e., R or S). Enantiomers may be characterized by their ability to rotate oncoming plane-polarized light to the right, designated as dextrorotatory, “(+)” or “D”, or to the left, designated as levorotatory, “(−)” or “L”. Enantiomers may exist as racemic mixtures or scalemic mixtures. Geometric isomers refer to isomers in which the spatial relationship of atoms around a double bond are different, typically designated E or Z according to conventional understanding in the chemical art. Geometric isomers may also exist as mixtures of E and Z isomers. All of the aforementioned isomeric variations of the chemical names or structures recited herein are included.

In the first aspect, the present disclosure relates to a fragrance composition comprising at least:
a) 2,3-butanediol and/or 1,3-butanediol, and
b) a fragrance component,
wherein the fragrance composition is free of ethanol and is a homogeneous and transparent solution.

The 2,3-butanediol and/or 1,3-butanediol may be synthesized according to known methods or, typically, obtained from commercial sources. Typically, the 2,3-butanediol and/or 1,3-butanediol are obtained from commercial sources. The grade and/or origin of 2,3-butanediol and/or 1,3-butanediol are not particularly limited.

In an embodiment, the fragrance composition comprises 2,3-butanediol and no 1,3-butanediol. In another embodiment, the fragrance composition comprises 1,3-butanediol and no 2,3-butanediol. In yet another embodiment, the fragrance composition comprises both 2,3-butanediol and 1,3-butanediol.

The amount of 2,3-butanediol is present in the fragrance composition is not particularly limited. However, in some embodiments, 2,3-butanediol is present in an amount from 1% to 95%, typically 5% to 70%, more typically 30% to 70%, by weight relative to the total weight of the fragrance composition.

The amount of 1,3-butanediol is present in the fragrance composition is not particularly limited. However, in some embodiments, 1,3-butanediol is present in an amount from 1% to 95%, typically 5% to 70%, more typically 30% to 70%, by weight relative to the total weight of the fragrance composition.

The amount of the fragrance component in the fragrance composition is not particularly limited. However, in an embodiment, the fragrance component is present in an amount from 1% to 99%, typically 5% to 95%, more typically 30% to 70%, by weight relative to the total weight of the fragrance composition.

The fragrance component may optionally comprise a perfumery solvent. A detailed description of the nature and type of solvents commonly used in perfumery, herein “perfumery solvent” cannot be exhaustive. However, exemplary solvents include, but are not limited to, solvents such as glycerol, dipropylene glycol and its monoether, 1,2,3-propanetriyl triacetate, dimethyl glutarate, dimethyl adipate 1,3-diacetyloxypropan-2-yl acetate, diethyl phthalate, isopropyl myristate, Abalyn® (rosin resins, available from Eastman), benzyl benzoate, benzyl alcohol, 2-(2-ethoxyethoxy)-1-ethanol, tri-ethyl citrate or mixtures thereof. Naturally derived solvents, like glycerol or various vegetable oils such as palm oil, sunflower oil or linseed oil, may also be used. Other non-limiting perfumery solvents include limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

The amount of the perfumery solvent is not particularly limited. However, in some embodiments, the perfumery solvent is present in an amount from 0% to 50%, typically 0.1% to 15%, more typically 0.2% to 10%, by weight relative to the total weight of the fragrance component.

The fragrance composition may be free of certain solvents.

As used herein, the phrase “free of” means that there is no external addition of the material modified by the phrase and that there is no detectable amount of the material that may be observed by analytical techniques known to the ordinarily-skilled artisan, such as, for example, gas or liquid chromatography, spectrophotometry, optical microscopy, and the like.

In an embodiment, the fragrance composition is free of solvents selected from the group consisting of glycerol, isobutyric acid 1-hydroxy-2,2,4-trimethyl-3-pentyl ester, isobutyric acid-3-hydroxy-2,2,4-trimethyl-1-pentyl ester, and combinations thereof.

The fragrance component of the fragrance composition may comprise one or more high olfactive impact ingredients. As used herein, a high olfactive impact ingredient is an ingredient that has been found to provide a high level olfactive impact in combination with the components of the fragrance composition.

Suitable high olfactive impact ingredients include, but are not limited to:
(+−)-1-methoxy-3-hexanethiol,
2-furanmethanethiol,
diallyl disulfide,
2-methoxy-3-(1-methylpropyl) pyrazine,
(+−)-2-(4-methyl-3-cyclohexen-1-yl)-2-propanethiol,
(Z)-2-nonenal,
2-(methylthiomethyl) furan,
1-(pyrazinyl)-1-ethanone,
methylmercaptan,
methyl 2-methyl-3-furyl disulfide,
5-methyl-2-hepten-4-one,
1-(1,3-thiazol-2-yl)-1-ethanone,
dimethyl trisulfide,
(+−)-3-mercaptohexyl acetate,
(Z)-4-heptenal,
3-methylbutanoic acid,
2-methoxy-3-(4-methylpentyl) pyrazine,
2,6,6-trimethyl-1,3-cyclohexadiene-1-carbaldehyde,
(+−)-4-hydroxy-2,5-dimethyl-3 (2h)-furanone,
3-ethyl-2,5-dimethylpyrazine,
2-ethyl-3,5-dimethylpyrazine,
(Z)-6-nonenal,
(Z)-4-decenal,
(2E,6Z)-2,6-nonadien-1-ol,
2-isopropyl-4-methylthiazole,
2-isopropyl-3-methoxypyrazine,
trimethylamine,
4-mercapto-4-methyl-2-pentanone,
(+−)-2-methylbutanoic acid,
(2Z,6Z)-2,6-nonadienenitrile,
(2E,6Z)-2,6-nonadienenitrile,
pyrazobutyle,
(+−)-2-methyldecanal,
(+−)-ethyl 2-methylpentanoate,
4-methylphenyl acetate,
(+−)-PERHYDRO-4α,8Aβ-dimethyl-4a-naphthalenol,
(+−)-ethyl 2-methylbutanoate,
(1R,5R)-4,7,7-trimethyl-6-thiabicyclo[3.2.1]oct-3-ene,
(1R,5R)-7,7-dimethyl-4-methylene-6-thiabicyclo[3.2.1]octane,
(1R,4R,5R)-4,7,7-trimethyl-6-thiabicyclo[3.2.1]octane,
(1R,5R)-4,7,7-trimethyl-6-thiabicyclo[3.2.1]oct-3-ene,
(1R,4R,5R)-4,7,7-trimethyl-6-thiabicyclo[3.2.1]octane,
2-ethoxy-4-methylphenol,
(3E,5Z)-1,3,5-undecatriene,
4-ethyl-2-methoxyphenol,
1-(4-methylphenyl) ethenone,
ethyl isobutyrate,
2-methoxyphenol,
(4E)-4-decenal,
(2E,6Z)-1,1-diethoxy-2,6-nonadiene,
4-methylphenol,
ethyl butanoate,
2-methoxy-3-methylpyrazine,
2-methoxy-6-methylpyrazine,
(1R,4R)-8-mercapto-3-p-menthanone,
butanoic acid,
3-(6,6-dimethyl-bicyclo[3.1.1]hept-2-en-2-yl) propanal,
(+−)-2-ethyl-4,4-dimethyl-1,3-oxathiane,
gamma octalactone,
1-(2-pyridyl)-1-ethanone,
allyl (3-methylbutoxy)acetate,
(+−)-allyl(2-methylbutoxy)acetate,
(2E,6Z)-2,6-nonadienal,
2-ethyl-3-methylpyrazine,
(+−)-2,6-dimethyl-5-heptenal,
(2E)-1-[(1RS,2SR)-2,6,6-trimethyl-3-cyclohexen-1-yl]-2-buten-1-one,
10-undecenal,
(9E)-9-undecenal,
(9Z)-9-undecenal,
2,3,5-trimethylpyrazine,
2,3-pentanedione,
2-hydroxy-3-methyl-2-cyclopenten-1-one,
4-methylphenyl isobutyrate,
1-decen-4-yne,
(+−)-(4Z)-4-cycloocten-1-yl methyl carbonate,
heptanal,
(+−)-cis-tetrahydro-methyl-4-methylene-6-phenyl-2 h-pyran,
(+−)-cis-3,6-dihydro-4,6-dimethyl-2-phenyl-2 h-pyran,
(+−)-cis-3,6-dihydro-2,4-dimethyl-6-phenyl-2 h-pyran,
(+−)-2-methylundecanal,
hexanal,
8-isopropyl-6-methyl-bicyclo[2.2.2]oct-5-ene-2-carbaldehyde,
ethyl hexanoate,
(+−)-1-octen-3-ol,
(+)-(2S)-2-methylbutanoic acid,
octanal,
2,6,6-trimethyl-2-cyclohexene-1,4-dione,
(1RS,2RS)-2,4-dimethyl-3-cyclohexene-1-carbaldehyde,
(1RS,2SR)-2,4-dimethyl-3-cyclohexene-1-carbaldehyde,
(+−)-ethyl 3-methyl-2-oxopentanoate,
(1RS,6RS)-3,6-dimethyl-3-cyclohexene-1-carbaldehyde,
(1RS,6RS)-4,6-dimethyl-3-cyclohexene-1-carbaldehyde,
(1RS,6SR)-4,6-dimethyl-3-cyclohexene-1-carbaldehyde,
2-hexyl-2-cyclopenten-1-one,
ethyl 4,6,6-trimethyl-1,3-cyclohexadiene-1-carboxylate,
3-phenylpropanal,
(−)-(2S,4R)-4-methyl-2-(2-methyl-1-propen-1-yl)tetrahydro-2H-pyran,
2-phenylethanol,
(+−)-methyl 2,2-dimethyl-6-methylidenecyclohexanecarboxylate,
(+−)-2,4-dimethyl-4-phenyltetrahydrofuran,
(E)-2-octenal,
ethyl benzoate,
(−)-(5R)-5-isopropenyl-2-methyl-2-cyclohexen-1-one,
methyl 2-octynoate,
2-phenylpropanal,
(2E)-2-hexenal,
(+−)-4-methoxy-2,5-dimethyl-3 (2H)-furanone,
methyl phenylacetate,
(2RS,4SR)-4-methyl-2-(2-methyl-1-propen-1-yl)tetrahydro-2H-pyran,
(2RS,4RS)-4-methyl-2-(2-methyl-1-propen-1-yl)tetrahydro-2H-pyran,
3-(4,4-dimethyl-1-cyclohexen-1-yl) propanal,
ethyl phenylacetate,
ethyl tricyclo[5.2.1.0. (2,6)]decane-2-carboxylate;
(+−)-5-ethyl-4-hydroxy-2-methyl-3 (2H)-furanone,
(+−)-2-ethyl-4-hydroxy-5-methyl-3 (2H)-furanone,
1-(2-aminophenyl)-1-ethanone,
(+−)-6-methyl-7-oxa-1-thia-4-azaspiro[4.4]nonane,
3-methylindole
7-methyl-2H-1,5-benzodioxepin-3 (4H)-one,
3-propylphenol,
(3RS,3aRS,6SR,7aSR)-3,6-dimethylhexahydro-1-benzofuran-2 (3H)-one,
(3RS,3aSR,6RS,7aRS)-3,6-dimethylhexahydro-1-benzofuran-2 (3H)-one,
(3aRS,5aRS,8aRS,8bSR)-2,2,6,6,7,8,8-heptamethyldecahydro-2H-indeno[4,5-b]furan
(4Z)-4-dodecenal,
(E)-2-ethoxy-5-(1-propenyl) phenol,
gamma octalactone,
1-(3-methyl-1-benzofuran-2-yl) ethenone,
(+−)-1-(5-propyl-1,3-benzodioxol-2-yl) ethenone,
(2S)-2-methyl-4-[(1R)-2,2,3-trimethyl-3-cyclopenten-1-yl]-4-penten-1-ol,
(2R)-2-methyl-4-[(1R)-2,2,3-trimethyl-3-cyclopenten-1-yl]-4-penten-1-ol,
methyl 2-aminobenzoate,
1-(3,3-dimethyl-1-cyclohexen-1-yl)-4-penten-1-one,
1-(5,5-dimethyl-1-cyclohexen-1-yl)-4-penten-1-one,
(3S,3aR,5R,8S,8aS)-5-isopropenyl-3,8-dimethyloctahydro-3a (1H)-azulenol,
(1S,3aR,4S,7R,8aS)-7-isopropenyl-1,4-dimethyldecahydro-4-azulenol,
(1S,3aR,4R,7R,8aS)-7-isopropenyl-1,4-dimethyldecahydro-4-azulenol,
(1R,3R,6S,7S,8S)-2,2,6,8-tetramethyltricyclo[5.3.1.0~3,8~]undecan-3-ol,
(1R,3R,6S,7S,8S)-3-ethoxy-2,2,6,8-tetramethyltricyclo[5.3.1.0~3,8~]undecane,
(E)-2-methoxy-4-(1-propenyl)phenyl acetate,
4-methoxybenzaldehyde,
5-nonanolide,
(+−)-(2,5-dimethyl-2,3-dihydro-1H-inden-2-yl) methanol,
3-methyl-4-octanolide,
(+−)-3-(4-hydroxy-4-methylpentyl)-3-cyclohexene-1-carbaldehyde,
(+−)-4-(4-hydroxy-4-methylpentyl)-3-cyclohexene-1-carbaldehyde,
(−)-(2E)-2-ethyl-4-[(1R)-2,2,3-trimethyl-3-cyclopenten-1-yl]-2-buten-1-ol,
4-(4-hydroxyphenyl)-2-butanone,
8-isopropylquinoline,
6-isopropylquinoline,
(3Z)-3,12-tridecadienenitrile,
(3E)-3,12-tridecadienenitrile,
(−)-(1R,3R,6S,7S,8S)-2,2,6,8-tetramethyltricyclo[5.3.1.0~3,8~]undecan-3-ol,
(+−)-1-(5-ethyl-5-methyl-1-cyclohexen-1-yl)-4-penten-1-one,
(4Z)-4-dodecenal,
(E)-3-phenyl-2-propenenitrile,
2-methoxy-4-(2-propen-1-yl) phenol,
(+−)-4-methylene-2-phenyltetrahydro-2H-pyran,
(+−)-4-methyl-6-phenyl-3,6-dihydro-2H-pyran,
(+−)-4-methyl-2-phenyl-3,6-dihydro-2H-pyran,
2,6-dimethoxyphenol,
(+−)-6-pentyltetrahydro-2H-pyran-2-one,
(3S,3aS,6R,7aR)-3,6-dimethylhexahydro-1-benzofuran-2 (3H)-one,
(3R,3aS,6R,7aR)-3,6-dimethylhexahydro-1-benzofuran-2 (3H)-one,
(+−)-2-methyl-3-[4-(2-methyl-2-propanyl)phenyl]propanal,
(−)-(1R,3S,7R,8R,10S,13R)-5,5,7,9,9,13-hexamethyl-4,6-dioxatetracyclo[6.5.1.0(1,10).0 (3,7)]tetradecane,
(4E,8E)-4,8-cyclododecadien-1-one,
(4E,8Z)-4,8-cyclododecadien-1-one,
(4Z,8E)-4,8-cyclododecadien-1-one,
2-methoxynaphthalene,
(4-methylphenoxy) acetaldehyde,
perhydro-2-chromenone,
(+)-2-{(1S)-1-[(1R)-3,3-dimethylcyclohexyl]ethoxy}-2-methylpropyl propionate,
methyl 2,4-dihydroxy-3,6-dimethylbenzoate,
(E)-3-methyl-5-(2,2,3-trimethyl-3-cyclopenten-1-yl)-4-penten-2-ol,
(2E)-2-dodecenal,
ethyl 2,3-epoxy-3-phenylbutanoate,
patchouli,
(+−)-(2E)-1-(2,6,6-trimethyl-2-cyclohexen-1-yl)-2-buten-1-one,
5-isopropyl-2-methylphenol,
2-(3-phenylpropyl)pyridine,
4-allyl-2-methoxyphenyl acetate,
4-hydroxy-3-methoxybenzaldehyde,
1-[(1RS,2RS)-1,2,8,8-tetramethyl-1,2,3,4,5,6,7,8-octahydro-2-naphthalenyl]ethenone,
1-((2RS,3RS)-2,3,8,8-tetramethyl-1,2,3,4,5,6,7,8-octahydronaphthalen-2-yl) ethenone,
1-[(2RS,3RS,8aRS)-2,3,8,8-tetramethyl-1,2,3,5,6,7,8,8a-octahydro-2-naphthalenyl]ethenone,
1-[(1RS,2RS,8aSR)-1,2,8,8-tetramethyl-1,2,3,5,6,7,8,8a-octahydro-2-naphthalenyl]ethenone,
1-[(2RS,3RS,8aRS)-2,3,8,8-tetramethyl-1,2,3,4,6,7,8,8a-octahydro-2-naphthalenyl]ethenone,
2-ethyl-3-hydroxy-4 (4H)-pyranone,
(2E)-1-(2,6,6-trimethyl-1,3-cyclohexadien-1-yl)-2-buten-1-one,
1-oxaspiro[4.5]decan-2-one,
3-[4-(2-hydroxy-2-methylpropyl)phenyl]propanal,
4-methylphenyl 3-methylbutanoate,
2-methyl-2-pentenoic acid,
7-isopropyl-2H,4H-1,5-benzodioxepin-3-one,
4-(4-methoxyphenyl)-2-butanone,
3-(4-tert-butylphenyl) propanal,
(−)-(3aR,5aS,9aS,9bR)-3a,6,6,9a-tetramethyldodecahydronaphtho[2,1-b]furan,
(+−)-5-heptyldihydro-2 (3H)-furanone,
2-methoxy-4-[(1E)-1-propen-1-yl]phenol,
(2E)-1-(2,6,6-trimethyl-1,3-cyclohexadien-1-yl)-2-buten-1-one,
(2Z)-3-methyl-5-phenyl-2-pentenenitrile,
(2E)-3-methyl-5-phenyl-2-pentenenitrile,
phenylacetic acid,
(+)-(1R,7R)-10,10-dimethyl-tricyclo[7.1.1.0 (2,7)]undec-2-en-4-one,
(+−)-4-ethyloctanoic acid,
(+−)-2,4-dimethyl-4,4a,5,9b-tetrahydroindeno[1,2-d][1,3]dioxine,
3-ethoxy-4-hydroxybenzaldehyde,
5-ethyl-3-hydroxy-4-methyl-2 (5H)-furanone,
3-(3,3-dimethyl-2,3-dihydro-1H-inden-5-yl) propanal,
3-(1,1-dimethyl-2,3-dihydro-1H-inden-4-yl) propanal,
3-(1,1-dimethyl-2,3-dihydro-1H-inden-5-yl) propanal,
(+−)-4-nonanolide,
ethyl (E)-3-phenyl-2-propenoate,
(+−)-(E)-3-methyl-4-(2,6,6-trimethyl-2-cyclohexen-1-yl)-3-buten-2-one,
3,7-dimethyl-6-octen-1-ol,
3,7-dimethyl-7-octen-1-ol,
1,5,9-trimethyl-4,8-decadienyl acetate,
indole,
3-butylidene-1-benzo[c]furanone,
thymol
1-(4-tert-butyl-3,5-dinitro-2,6-dimethylphenyl)-1-ethanone,
   or any mixture thereof.

In an embodiment, the fragrance component comprises one or more high olfactive impact ingredients selected from the group consisting of:
2,6,6-trimethyl-1,3-cyclohexadiene-1-carbaldehyde,
(3E,5Z)-1,3,5-undecatriene,
(4E)-4-decenal,
(2E)-1-[(1RS,2SR)-2,6,6-trimethyl-3-cyclohexen-1-yl]-2-buten-1-one, octanal,
(1RS,2RS)-2,4-dimethyl-3-cyclohexene-1-carbaldehyde,
(1RS,2SR)-2,4-dimethyl-3-cyclohexene-1-carbaldehyde,
(+−)-methyl 2,2-dimethyl-6-methylidenecyclohexanecarboxylate,
ethyl benzoate,
(−)-(5R)-5-isopropenyl-2-methyl-2-cyclohexen-1-one,
4-hydroxy-3-methoxybenzaldehyde,
(−)-(3aR,5aS,9aS,9bR)-3a,6,6,9a-tetramethyldodecahydronaphtho[2,1-b]furan,
(+−)-2,4-dimethyl-4,4a,5,9b-tetrahydroindeno[1,2-d][1,3]dioxine,
(+−)-4-nonanolide,
3,7-dimethyl-6-octen-1-ol,
indole, and any mixture thereof.

The fragrance component may be characterized by an average log P, which is the average of the log P values of the ingredients of the fragrance component. As used herein, “log P” refers to the logarithm (base 10) of the partition coefficient (P), which is defined as the ratio of a compound's organic (typically, oil)-to-aqueous phase concentrations. Log P describes the partition of a compound in a two-phase system made of octanol and water (Log P=Log (C_{oct}/C_{water})). This parameter provides a measure of the hydrophilic/lipophilic character of a compound: the higher the Log P, the more lipophilic the compound. Log P values may be determined empirically or calculated. In some embodiments, the log P values of the ingredients of the fragrance component are calculated. Calculated log P, or C log P, may be obtained for each single perfuming ingredient according to methods known to those of ordinary skill in the art. For example, C log P can be obtained according to the program EPI suite (4.0); EPA (US Environmental Protection Agency) and Syracuse Research Corporation (SRC), 2000. In another example, C log P may be calculated according to a method described by Suzuki T. 1992, CHEMICALC 2, QCPE Program No 608, Department of chemistry, Indiana University; Suzuki T., Kudo Y. J. Comput.-Aided Mol. Design 1990, 4, 155; Suzuki T., J. Comput.-Aided Mol. Design 1991, 5, 149. In yet another example, C log P may be determined using an application available at the following website: http://www.daylight.com/daycgi/clogp.

In an embodiment, the fragrance component has an average C log P greater than 4.

In another embodiment, the fragrance component comprises at least 20% by weight of ingredients having C log P between 4 and 5, and at least 20% by weight of ingredients having C log P between 5 and 6, relative to the weight of the fragrance component.

The fragrance composition may further comprise one or more solvents characterized by a log P greater than or equal to −2 and less than or equal to 2.

In an embodiment, the fragrance composition further comprises one or more solvents characterized by a log P greater than or equal to −2 and less than or equal to 2 selected from the group consisting of glycols, typically 1,2-alkanediols, such as 1,2-propanediol, 1,2-butanediol, 1,2-pentanediol, 1,2-hexanediol, 1,2-heptanediol, and 1,2-octanediol, and 1,3-alkanediols, such as 1,3-propanediol, 2-methyl-1,3-propanediol, and 3-methyl-1,3-butanediol; polyalkylene glycols, typically polyethylene glycols, polypropylene glycols, and poly(ethylene/propylene) glycols; C₁-C₁₀ alkyl esters of citric acid, typically triethyl citrate; and C₃-C₁₀ alkanols.

In an embodiment, the fragrance composition further comprises a 1,2-alkanediol, typically 1,2-pentanediol and/or 1,2-octanediol. In an embodiment, the fragrance composition further comprises a 1,2-alkanediol selected from the group consisting of 1,3-propanediol, 1,2-pentanediol, 1,2-octanediol, and mixtures thereof. In another embodiment, the fragrance composition further comprises 1,3-propanediol, 1,2-pentanediol, and 1,2-octanediol.

The amount of the one or more solvents characterized by a log P greater than or equal to −2 and less than or equal to 2 is not particularly limited. However, in an embodiment, the one or more solvents characterized by a log P greater than or equal to −2 and less than or equal to 2 is present in an amount of from 0.1% to 50%, typically 0.1% to 30%, more typically 5% to 15%, by weight relative to the total weight of the fragrance composition.

The fragrance composition may further comprise water. The form of water used is not particularly limited. Water used according to the present disclosure may be deionized and/or demineralized. Suitable water may be water obtained by various extraction methods known to those of ordinary skill in the art, such as distillation, or from natural water sources, such as glaciers, springs, seas and oceans, aquifers, and the like.

In an embodiment, water is present in an amount from 0.1% to 99%, typically 20% to 70%, more typically 40% to 60%, by weight relative to the total weight of the fragrance composition.

In an embodiment in which water is present, 2,3-butanediol is present in an amount from 1% to 90%, typically 5% to 40%, more typically 10% to 30%, by weight relative to the total weight of the fragrance composition.

In another embodiment in which water is present, 1,3-butanediol is present in an amount from 1% to 90%, typically 5% to 40%, more typically 10% to 30%, by weight relative to the total weight of the fragrance composition.

In yet another embodiment in which water is present, the fragrance component is present in an amount from 1% to 50%, typically 1% to 20%, more typically 1% to 10%, by weight relative to the total weight of the fragrance composition.

The present disclosure contemplates solubilizing high dosages of fragrances to obtain aesthetically pleasing transparent products without the need for any emulsions, surfactants, or other solubilizers.

Thus, in some embodiments, the fragrance composition is free of any emulsions, surfactants, or other solubilizers. In an embodiment, the fragrance composition is free of any emulsions and/or surfactants.

The fragrance composition may further comprise a perfuming co-ingredient. As used herein, a perfuming co-ingredient refers to ingredients that impart a hedonic effect, i.e., used for the primary purpose of conferring or modulating an odor. In other words, such a co-ingredient, to be considered as being a perfuming one, must be recognized by a person skilled in the art as being able to impart or modify in a positive or pleasant way the odor of a composition, and not just as having an odor.

Perfuming co-ingredients may impart an additional benefit beyond that of modifying or imparting an odor, such as long-lasting, blooming, malodour counteraction, antimicrobial effect, antiviral effect, microbial stability, or pest control.

The nature and type of the perfuming co-ingredients do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds, and essential oils. Perfuming co-ingredients can be of natural or synthetic origin. Suitable perfumery co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release, in a controlled manner, various types of perfuming compounds, known as properfume or profragrance.

The fragrance composition may optionally comprise a fragrance modulator.

Fragrance modulators, also known as fixatives, are agents having the capacity to affect the manner in which the odor, and in particular the evaporation rate and intensity, of the compositions incorporating said modulator can be perceived by an observer or user thereof, over time, as compared to the same perception in the absence of the modulator. In particular, the modulator allows prolonging the time during which their fragrance is perceived.

Examples of fragrance modulators suitable for use according to the present disclosure include, but are not limited to, caprylyl alcohol, octanol, butyloctanol, isotridecyl alcohol, hexyldecanol, isostearyl alcohol, octyldecanol, octyldodecanol, decyltetradecanol, tetradecyloctadecanol, PPG-20 methyl glucose ether, methyl glucoside polyol; ethyl glucoside polyol; propyl glucoside polyol; isocetyl alcohol; PPG-3 myristyl ether; neopentyl glycol diethylhexanoate; sucrose laurate; sucrose dilaurate, sucrose myristate, sucrose palmitate, sucrose stearate, sucrose distearate, sucrose tristearate, hyaluronic acid disaccharide sodium salt, sodium hyaluronate, propylene glycol propyl ether; dicetyl ether; polyglycerin-4 ethers; isoceteth-5; isoceteth-7, isoceteth-10; isoceteth-12; isoceteth-15; isoceteth-20; isoceteth-25; isoceteth-30; disodium lauroamphodipropionate; hexaethylene glycol monododecyl ether; and their mixtures; neopentyl glycol diisononanoate; cetearyl ethylhexanoate; panthenol ethyl ether, DL-panthenol, N-hexadecyl n-nonanoate, noctadecyl n-nonanoate, a profragrance, cyclodextrin, an encapsulation, and any combination thereof.

The fragrance composition described herein may comprise a solid carrier. The fragrance composition or some element of the fragrance composition, such as the fragrance component, can be chemically or physically bound. In general, such solid carriers are employed either to stabilize the composition, or to control the rate of evaporation of the compositions or of some ingredients. Solid carriers are of current use in the art and a person skilled in the art knows how to reach the desired effect. Suitable solid carriers include, but are not limited to, absorbing gums or polymers or inorganic materials, such as porous polymers, cyclodextrines, dextrines, maltodextrines wood-based materials, organic or inorganic gels, clays, gypsum talc or zeolites.

Other suitable solid carriers include encapsulating materials. Examples of such materials may comprise wall-forming and plasticizing materials, such as glucose syrups, natural or modified starches, hydrocolloids, cellulose derivatives, polyvinyl acetates, polyvinylalcohols, proteins or pectins, plant gums such as acacia gum (Gum Arabic), urea, sodium chloride, sodium sulphate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, carbohydrates, saccharides such as sucrose, mono-, di-, and polysaccharides and derivatives such as chitosan, starch, cellulose, carboxymethyl methylcellulose, methylcellulose, hydroxyethyl cellulose, ethyl cellulose, propyl cellulose, polyols/sugar alcohols such as sorbitol, maltitol, xylitol, erythritol, and isomalt, polyethylene glycol (PEG), polyvinyl pyrrolidin (PVP), polyvinyl alcohol, acrylamides, acrylates, polyacrylic acid and related, maleic anhydride copolymers, amine-functional polymers, vinyl ethers, styrenes, polystyrenesulfonates, vinyl acids, ethylene glycol-propylene glycol block copolymers, vegetable gums, gum acacia, pectins, xanthanes, alginates, carragenans, citric acid or any water soluble solid acid, fatty alcohols or fatty acids and mixtures thereof.

Other suitable encapsulating materials are described in reference texts known to those of skill in the art, such as H. Scherz, Hydrokolloide: Stabilisatoren, Dickungs-und Geliermittel in Lebensmitteln, Band 2 der Schriftenreihe Lebensmittelchemie, Lebensmittelqualität, Behr's Verlag Gmbh & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

Other exemplary solid carriers include core-shell capsules with resins of aminoplast, polyamide, polyester, polyurea or polyurethane type, and mixtures thereof, made using techniques well-known to those of ordinary skill in the art, such as phase separation induced by polymerization, interfacial polymerization, coacervation, or a combination thereof, optionally in the presence of a polymeric stabilizer or of a cationic copolymer.

Resins may be produced by the polycondensation of an aldehyde (e.g., formaldehyde, 2,2-dimethoxyethanal, glyoxal, glyoxylic acid or glycolaldehyde, and mixtures thereof) with an amine such as urea, benzoguanamine, glycoluryl, melamine, methylol melamine, methylated methylol melamine, guanazole and the like, as well as mixtures thereof. Alternatively, one may use preformed resins like alkylolated polyamines such as those commercially available under the trademark Urac® (origin: Cytec Technology Corp.), Cymel® (origin: Cytec Technology Corp.), Urecoll® or Luracoll® (origin: BASF).

Other suitable resins are the those produced by the polycondensation of a polyol, like glycerol, and a polyisocyanate, for example, a trimer of hexamethylene diisocyanate, a trimer of isophorone diisocyanate or xylylene diisocyanate or a Biuret of hexamethylene diisocyanate or a trimer of xylylene diisocyanate with trimethylolpropane (marketed as Takenate® by Mitsui Chemicals), among which a trimer of xylylene diisocyanate with trimethylolpropane and a Biuret of hexamethylene diisocyanate are of mention.

The encapsulation of perfumes by polycondensation of amino resins, namely melamine-based resins with aldehydes is well-known in the art. Pertinent publications include, but are not limited to, K. Dietrich et al. Acta Polymerica, 1989, vol. 40, pages 243, 325 and 683, as well as 1990, vol. 41, page 91 and U.S. Pat. No. 4,396,670 issued Aug. 2, 1983. The general knowledge in encapsulation technology is very significant and cannot be exhaustive. More recent publications of pertinence, which disclose suitable uses of such microcapsules, are represented, for example, by the article of K. Bruyninckx and M. Dusselier, ACS Sustainable Chemistry & Engineering, 2019, vol. 7, pages 8041-8054. These publications are incorporated herein by reference.

The fragrance composition may optionally comprise at least one perfumery adjuvant.

The at least one perfumery adjuvant is an ingredient capable of imparting an additional added benefit such as a color, a particular light resistance, chemical stability, etc. A detailed description of the nature and type of adjuvant commonly used in perfuming compositions cannot be exhaustive, but it has to be mentioned that said ingredients are well known to a person skilled in the art.

Exemplary perfumery adjuvants include, but are not limited to, viscosity agents (e.g., surfactants, thickeners, gelling and/or rheology modifiers), stabilizing agents (e.g., preservatives, antioxidant, heat/light and or buffers or chelating agents, such as BHT), coloring agents (e.g., dyes and/or pigments), preservatives (e.g. antibacterial or antimicrobial or antifungal or anti irritant agents), abrasives, skin cooling agents, insect repellants, ointments, vitamins and mixtures thereof.

The fragrance compositions according to the present disclosure may be prepared according to any method known to those of ordinary skill in the art. The ordinarily-skilled artisan is perfectly able to design optimal formulations for the desired effect by admixing the above mentioned components to arrive at the desired composition by applying standard knowledge and concepts known to those of ordinary skill and by utilizing routine optimization methodologies.

According to the present disclosure, the fragrance composition is a homogeneous and transparent solution. As used herein, the term “homogeneous” means that all components of the composition are completely solubilized and the composition is uniform throughout. As used herein, the term “transparent” means that the composition has the property of transmitting light without appreciable light scattering. Transparency of a composition can be assessed by determining its turbidity expressed in NTU (Nephelometric Turbidity Units) using a turbidimeter, measured in a 2.5 cm cell at 25 °C, at wavelengths between 400 and 600 nm. In some embodiments, the composition has a turbidity comprised between 0 and 20 NTU.

In the second aspect, the present disclosure relates to a consumer product comprising the fragrance composition described herein.

The form of the consumer product is not particularly limited. In an embodiment, the consumer product is a perfume, a body-care product, a cosmetic preparation, a skin-care product, a fabric care product, an air care product, or a home care product.

In another embodiment, the consumer product is a fine perfume, a splash, an eau de toilette, an eau de parfum, a cologne, a body mist, a body spray, a hair mist, a shave or after-shave lotion, a shampoo, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, a foot/hand care products, a hygiene product, a liquid or solid or unit-dose detergent, a fabric softener, a solid or liquid fabric scent-booster, a fabric refresher, an ironing water, an air freshener, a “ready to use” powdered air freshener, a mold remover, a furniture care product, a wipe, a dish detergent or hard-surface detergent, a leather care product, or a car care product.

In an embodiment, the consumer product comprises the fragrance composition in an amount from 1% to 95%, typically from 2% to 80%, more typically from 3% to 70%, by weight relative to the total weight of the consumer product.

In an embodiment, the consumer product comprises the fragrance composition in an amount from 1% to 30%, typically from 2% to 20%, more typically from 3% to 10%, by weight relative to the total weight of the consumer product.

In the third aspect, the present disclosure relates to a method for enhancing or modulating the perceived olfactive impact and/or long lastingness of a fragrance composition, the method comprising adding 2,3-butanediol and/or 1,3-butanediol to a fragrance component, in the absence of ethanol, to obtain the fragrance composition as a homogeneous and transparent solution.

Generally, combining 2,3-butanediol and/or 1,3-butanediol with the fragrance component may be achieve using any suitable method known to those of ordinary skill in the art. For example, the components may be weighed and then mixed, typically by stirring, until homogeneous. No ethanol is added.

Mentioned is made of the use of 2,3-butanediol and/or 1,3-butanediol to enhance or modulate the perceived olfactive impact and/or long lastingness of a fragrance composition comprising a fragrance component.

The compositions, products, methods, and uses, according to the present disclosure are further illustrated by the following non-limiting examples.

### Example 1. Eau De Toilettes (EDTs) According to the Present Disclosure

Eau de Toilettes (EDTs) according to the present disclosure were made by combining a fragrance (“Fragrance A”) with the components and amounts summarized in Table 1 below. To obtain the compositions of the present disclosure, all organic solvents were weighed and mixed in a beaker under magnetic stirring. Fragrance, typically in the form of fragrance oil, was added to the mixture. Water was added and the solution was stirred until homogeneous.

TABLE 1

| | Control1 | EDT1 | EDT2 | EDT3 | EDT4 |
|---|---|---|---|---|---|
| Component | (wt %) | (wt %) | (wt %) | (wt %) | (wt %) |
| 2,3-butanediol¹⁾ | — | 10 | 10 | 20 | 22.5 |
| 1,3-propanediol²⁾ | — | 10 | — | — | — |
| 1,2-pentanediol³⁾ | — | 10 | 10 | 10 | — |
| 1,2-octanediol⁴⁾ | — | 10 | 10 | 10 | 22.5 |
| 1,3-butanediol⁵⁾ | — | — | 10 | — | — |
| Fragrance A | 10 | 10 | 10 | 10 | 10 |
| Ethanol | 79 | — | — | — | — |
| Water | 11 | 50 | 50 | 50 | 45 |

¹⁾2,3-butanediol; origin: GS CALTEX Corporation
²⁾1,3-Propanediol; origin: DuPont Tate & Lyle Bio Products
³⁾Pentylene Glycol; origin: Minasolve
⁴⁾Caprylyl Glycol; origin: Inolex
⁵⁾1,3-butylene glycol; origin: Genomatica

“Fragrance A” had an inverted pyramid structure and contained the perfumery ingredients listed in Table 2 below.

TABLE 2
Fragrance A ingredients
decanal
octanal
1-[(2RS,3RS,8aRS)-2,3,8,8-tetramethyl-1,2,3,5,6,7,8,8a-
octahydro-2-naphthalenyl] ethanone
benzyl acetate
(−)-(5R)-5-isopropenyl-2-methyl-2-cyclohexen-1-one
(2Z)-3,7-dimethyl-2,6-octadienal
(+−)-3,7-dimethyl-6-octen-1-ol
(+−)-3-methyl-5-(2,2,3-trimethyl-3-cyclopenten-1-yl)-2-pentanol
ethyl (2E,4Z)-2,4-decadienoate
(3E,5Z)-1,3,5-undecatriene
lavender oil
1-methyl-4-(prop-1-en-2-yl)cyclohex-1-ene
(+−)-4-isopropenyl-1-methylcyclohexene
(−)-(3R)-3,7-dimethyl-1,6-octadien-3-ol
(+-)-3,7-dimethyl-1,6-octadien-3-yl acetate
methyl 2-(methylamino)benzoate
petitgrain oil
[2-[1-(3,3-dimethylcyclohexyl)ethoxy]-2-oxoethyl] propanoate
(1RS,2SR,4RS)-2-methoxy-4-propylcyclohexanol
dipropylene glycol

### Example 2. Sensory Panel

A sensory panel was performed in order to compare the reference EDT (“Control1”) and inventive EDT (“EDT1”), both of which contain Fragrance A, to measure olfactive performance.

A Prazitherm PZ72 slide warmer was pre-heated to 32 °C for 30 minutes. Blotters were placed on the precision hotplate. Using an adjustable volume pipette, 20 μl of solution was dosed directly to the center of the blotter and evaporated at 32 °C. At different times (t=0 min (Fresh), 2 hours, 4 hours and 6 hours), the randomized blotters were evaluated by 7 panelists.

The methodology used is a 3-Alternative Forced Choice test. For each time point, panelists were presented with 3 samples, two of which were the Reference EDT (“Control1”) and one of which was inventive EDT1.

Panelists indicated the sample(s) that they perceived higher in terms of overall intensity.

### Hypothesis:

H0: The two samples are not different.

H1: The sample with technology is more intense than the sample without technology, in terms of overall intensity.

### Associated Risks:

H0 rejected=a risk:
Risk associated with a false alarm, concluding that products differ when in fact they do not.
Data was analyzed using the binomial statistic.

### Data Interpretation:

If the p-value obtained for α≤0.05, then the sample with technology was more intense in overall intensity than the sample without technology,
If the p-value obtained for α is 0.05<α≤0.10, then a trend difference was determined,
If the p-value obtained for α>0.10, the samples were not significantly different.

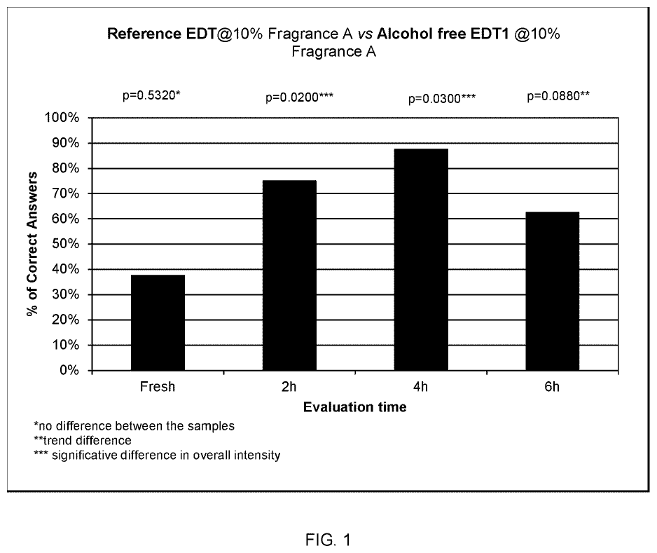

The result of the sensory evaluation is shown in FIG. 1. FIG. 1 shows a comparison of the olfactive performance between a reference EDT (“Control1”) and inventive EDT (“EDT1”), both of which contain the same fragrance. According to the results of the sensory panel presented in FIG. 1, after 2 hours and 4 hours of evaporation, the alcohol-free formulation containing 2,3-butanediol exhibited greater fragrance intensity than the classical Eau de Toilette formulation containing the same inverted pyramid fragrance. Moreover, the note is more Citrus and fresher with the fragrance evaluated in the alcohol-free formula. Top notes were also prolonged. Thus, it is shown that the alcohol-free formula containing 2,3-butanediol improved long-lastingness in comparison to the reference classical Eau de Toilette formulation.

### Example 3. Evaporation Kinetics

Two formulations, one comparative (“Control2”) and one inventive (“EDT5”), were made by combining a fragrance (“Fragrance B”) with the components and amounts summarized in Table 3 below.

TABLE 3

| | Control2 | EDT5 |
|---|---|---|
| Component | (wt %) | (wt %) |
| 2,3-butanediol¹⁾ | — | 10 |
| 1,3-propanediol²⁾ | — | 10 |
| 1,2-pentanediol³⁾ | — | 10 |
| 1,2-octanediol⁴⁾ | — | 10 |
| Fragrance B | 10 | 10 |
| Ethanol | 79 | — |
| Water | 11 | 50 |

¹⁾2,3-butanediol; origin: GC CALTEX
²⁾1,3-Propanediol; origin: DuPont Tate & Lyle Bio Products
³⁾Pentylene Glycol; origin: Minasolve
⁴⁾Caprylyl Glycol; origin: Inolex

“Fragrance B” contained the perfumery ingredients listed in Table 4 below.

TABLE 4
Fragrance B ingredients
(Z)-3-hexenyl acetate
allyl hexanoate
(1RS,2RS)-2,4-dimethyl-3-cyclohexene-1-carbaldehyde (A) +
(1RS,2SR)-2,4-dimethyl-3-cyclohexene-1-carbaldehyde (B)
methyl (1RS,2SR)-2,6,6-trimethyl-3-cyclohexene-1-carboxylate (A) +
methyl (1RS,2RS)-2,6,6-trimethyl-3-cyclohexene-1-carboxylate (B)
(2RS,4SR)-2-methyl-4-propyl-1,3-oxathiane (A) + (2RS,4RS)-2-methyl-
4-propyl-1,3-oxathiane (B)
(3Z)-3-hexen-1-yl butyrate
methyl benzoate
(+−)-methyl 2,2-dimethyl-6-methylidenecyclohexanecarboxylate
ethyl benzoate
2,6-dimethyl-2-heptanol
(Z)-3-hexenyl isobutyrate
(−)-propyl (S)-2-(1,1-dimethylpropoxy)propanoate
(3E,5Z)-1,3,5-undecatriene
2,6,6-trimethyl-1,3-cyclohexadiene-1-carbaldehyde
(+−)-3,7-dimethyl-1,6-octadien-3-ol
(1R,2R)-1,7,7-trimethyl-bicyclo[2.2.1]hept-2-yl acetate
(2RS,5SR)-5-methyl-2-(2-propanyl)cyclohexanone (A) +
(2RS,5RS)-5-methyl-2-(2-propanyl)cyclohexanone (B)
allyl heptanoate
(+)-(R)-3,7-dimethyl-6-octenal
(E)-4-decenal
(3Z)-1,3-undecadien-5-yne (A) + (3E)-1,3-undecadien-5-yne (B)
(+−)-(3Z)-3,4,5,6,6-pentamethyl-3-hepten-2-one (A) + (+-)-
3,5,6,6-tetramethyl-4-methylidene-2-heptanone (B) + (+-)-(3E)-
3,4,5,6,6-pentamethyl-3-hepten-2-one (C) + (+-)-(E)-3,4,5,6,6-
pentamethylhept-4-en-2-one (D)
1-methoxy-4-(2-propen-1-yl)benzene
2-phenylethyl formate
(E)-3,7-dimethyl-1,6-nonadien-3-ol (A) + (Z)-3,7-dimethyl-1,6-nonadien-
3-ol (B)
(+−)-2-pentylcyclopentanone
(2E)-1-[(1RS,2SR)-2,6,6-trimethyl-3-cyclohexen-1-yl]-2-buten-1-
one
3,7-dimethyl-6-octenyl acetate
(2Z)-3,7-dimethyl-2,6-octadien-1-yl acetate
methyl 2-nonynoate
(E)-3,7-dimethyl-2,6-octadienyl acetate
allyl 3-cyclohexylpropanoate
indole
(+-)-2,4-dimethyl-4,4a,5,9b-tetrahydroindeno[1,2-d][1,3]dioxine
(ISOMER A) + (+−)-2,4-dimethyl-4,4a,5,9b-tetrahydroindeno[1,2-d]
[1,3]dioxine (ISOMER B) (A + B)
(+−)-4-nonanolide
(+-)-3-(4-isopropylphenyl)-2-methylpropanal
(+−)-4-decanolide
6-hexyltetrahydro-2H-pyran-2-one
(+−)-(4E)-3-methyl-4-cyclopentadecen-1-one (A) + (+−)-(5E)-3-methyl-
5-cyclopentadecen-1-one (B) + (+−)-(5Z)-3-methyl-5-cyclopentadecen-
1-one (C)
(−)-(3aR,5aS,9aS,9bR)-3a,6,6,9a-
tetramethyldodecahydronaphtho[2, 1-b]furan
4-hydroxy-3-methoxybenzaldehyde
(+)-methyl (1R)-cis-3-oxo-2-pentyl-1-cyclopentaneacetate
2-chromenone
benzyl 2-hydroxybenzoate
dipropylene glycol

The evaporation kinetics of inventive EDT5 were compared to that of comparative formulation Control2.

Evaporations were done in Tzero lids. Prazitherm PZ72 slide warmer was pre-heated to 32 °C for 30 minutes. Each crucible was placed on the precision hotplate. Using an adjustable volume pipette, 10 μL of fragrance formulation was dosed directly to the center of the crucible and evaporated at 32 °C for 5 minutes (considered as the Time Zero, “TO”), 15 minutes, 1 hour, 2 hours, 4 hours and 6 hours on the precision hotplate. A triplicate set was performed for each sample and each condition tested. When time points were reached, each crucible was placed in a 2-mL Agilent GC vial (Agilent 5183-2068) and 500 μL ethanol was added to stop the evaporation. Vials were closed and mixed by shaking for at least 1 minute. Samples were analyzed by GC-MS direct injection methodology.

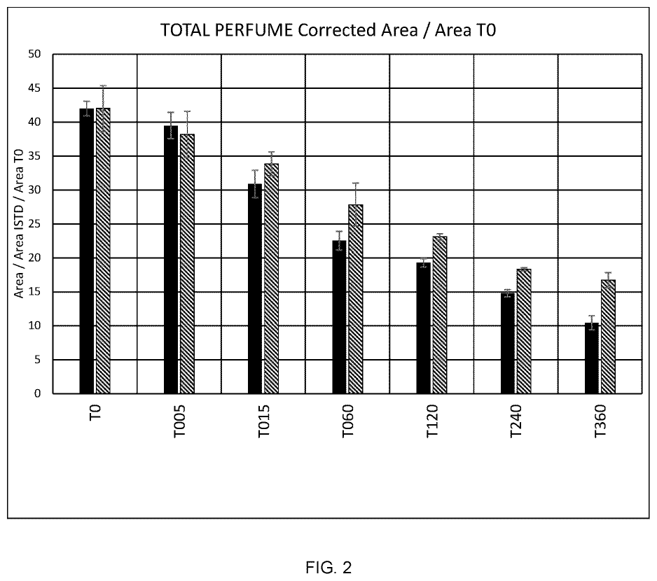

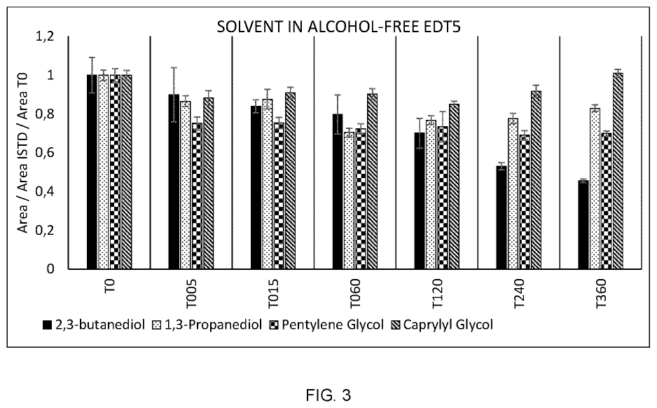

The results are shown in FIG. 2 and FIG. 3. FIG. 2 shows the evaporation rate of the fragrance component of the comparative and inventive formulations. FIG. 3 shows the evaporation rate of the solvent component of the comparative and inventive formulations. As shown in FIG. 2, the results of the evaporation kinetics evaluation highlight the ability of the alcohol-free perfume formula containing 2,3-butanediol (pattern fill) to improve long-lastingness by reducing evaporation rate of the fragrance component. The effect was perceptible after 15 min of evaporation. Interestingly, as shown in FIG. 3, the area of solvents in the inventive alcohol-free formula shows that 2,3-butanediol itself is evaporated more quickly than the other diols.

### Example 4. Solubility

The solubilities of various fragrance components having different proportions of ingredients of varying log P were determined for different ethanol-free bases. Generally, an ethanol-free base was charged into a beaker. While under magnetic stirring, a fragrance component was added drop by drop until appearance of non-homogeneity and/or turbidity. The mass of fragrance component added was noted and the solubility was calculated. The ethanol-free bases, including a comparative base, are summarized in Table 5a and 5b and the fragrance components are summarized in Table 6 below.

TABLE 5a

| | Control3 | ZGCP | GCP | BGCP | BGCP2 |
|---|---|---|---|---|---|
| Component | (wt %) | (wt %) | (wt %) | (wt %) | (wt %) |
| 2,3- | — | 11.1 | 22.2 | 11.1 | 16.7 |
| butanediol¹⁾ | | | | | |
| 1,3- | — | 11.1 | — | — | — |
| propanediol²⁾ | | | | | |
| 1,2- | 23.6 | 11.1 | 11.1 | 11.1 | 11.1 |
| pentanediol³⁾ | | | | | |
| 1,2- | 5.9 | 11.1 | 11.1 | 11.1 | 11.1 |
| octanediol⁴⁾ | | | | | |
| 1,3- | — | — | — | 11.1 | 5.6 |
| butanediol⁵⁾ | | | | | |
| Glycerol | 8.1 | — | — | — | — |
| Water | 62.4 | 55.6 | 55.6 | 55.6 | 55.6 |

¹⁾2,3-butanediol; origin: GS CALTEX Corporation
²⁾1,3-Propanediol; origin: DuPont Tate & Lyle Bio Products
³⁾Pentylene Glycol; origin: Minasolve
⁴⁾Caprylyl Glycol; origin: Inolex
⁵⁾1,3-butylene glycol; origin: Genomatica

TABLE 5b

| | Control4 | BCP |
|---|---|---|
| Component | (wt %) | (wt %) |
| 1,3-propanediol²⁾ | 22.2 | — |
| 1,2-pentanediol³⁾ | 11.1 | 11.1 |
| 1,2-octanediol⁴⁾ | 11.1 | 11.1 |
| 1,3-butanediol⁵⁾ | — | 22.2 |
| Water | 55.6 | 55.6 |

²⁾1,3-Propanediol; origin: DuPont Tate & Lyle Bio Products
³⁾Pentylene Glycol; origin: Minasolve
⁴⁾Caprylyl Glycol; origin: Inolex
⁵⁾1,3-butylene glycol; origin: Genomatica

TABLE 6

| Fra- | | | | | | | Other |
|---|---|---|---|---|---|---|---|
| grance | Log | 2<Log | 3<Log | 4<Log | 5<Log | Log | ingred- |
| com- | P <= 2 | P <= 3 | P <= 4 | P <= 5 | P <= 6 | P > 6 | ients |
| ponent | (%) | (%) | (%) | (%) | (%) | (%) | (%) |
| F1 | 0.00 | 0.06 | 0.06 | 0.47 | 14.56 | 1.85 | 83.00 |
| F2 | 22.90 | 10.64 | 1.05 | 14.72 | 3.66 | 8.26 | 38.75 |
| F3 | 2.57 | 36.94 | 18.89 | 11.78 | 3.48 | 0.10 | 26.25 |
| F4 | 21.12 | 21.25 | 8.08 | 8.31 | 27.00 | 8.78 | 5.46 |
| F5 | 3.90 | 8.68 | 23.50 | 23.84 | 25.07 | 4.53 | 10.47 |
| F6 | 0.40 | 17.26 | 15.59 | 13.26 | 22.34 | 10.77 | 20.38 |

9 g of an ethanol-free base was charged into a beaker. While under magnetic stirring, a fragrance component was added drop by drop until appearance of non-homogeneity and/or turbidity. The solubility results are shown in Table 7a and 7b below.

TABLE 7a
Solubility

| Fragrance | Average | | | | | |
|---|---|---|---|---|---|---|
| component | Log P | GCP | BGCP | BGCP-2 | ZGCP | Control3 |
| F1 | 0.35 | NT | NT | 20.0 | NT | 20 |
| F2 | 2.18 | 20.0 | 20.0 | 19.0 | 15.0 | 20 |
| F3 | 3.16 | 10.0 | 10.7 | 11.0 | 7.0 | 11 |
| F4 | 3.74 | NT | NT | 8.0 | NT | 8 |
| F5 | 4.36 | 7.5 | 7.5 | 7.5 | 5.7 | 2.4 |
| F6 | 3.90 | NT | NT | 7.0 | NT | 7 |

NT = not tested

TABLE 7b
Solubility

| Fragrance | Average | | |
|---|---|---|---|
| component | Log P | Control4 | BCP |
| F1 | 0.35 | 19.2 | 20.4 |
| F2 | 2.18 | 13.0 | 17.1 |
| F3 | 3.16 | 4.2 | 11.2 |
| F4 | 3.74 | 6.2 | 9.8 |
| F5 | 4.36 | 3 | 7.5 |
| F6 | 3.90 | 3.8 | 8.7 |

Notably, as shown in Table 7a, the solubility of F5 in the inventive formulations, which contained 2,3-butanediol and optionally 1,3-butanediol, was significantly higher than in the control formulation. Similarly, as shown in Table 7b, the solubility of F5 in the inventive formulation, which contained 1,3-butanediol, was significantly higher than in the control formulation, which did not contain 1,3-butanediol. This indicates that the inventive formulations provide higher solubility for fragrance components that have an average log P of 4 or greater.

Better solubility of the fragrances is obtained with all of the inventive formulas (GCP, BGCP, BGCP-2, ZGCP, BCP) compared to control4 which does not contain 2,3-butanediol or 1,3-butanediol. Addition of 2,3-butanediol or 1,3-butanediol improves the solubility of the fragrance. The Log P is an important parameter for solubilization. A fragrance having a high Log P is expected to be more challenging to solubilize into a water-based formula. The inventive formulas were able to solubilize much more fragrance than control3 and control4 as in the case of F5 which has the highest average Log P (>4).

### Example 5. Exemplary Compositions

Exemplary compositions (BW, BGW and GW) according to the present disclosure were made by combining phenylethyl alcohol, which is a commonly used perfumery raw material, using the components and amounts summarized in Table 8 below. To obtain the compositions, solvents were weighed and mixed in a beaker under magnetic stirring. Phenylethyl alcohol was added to the mixture. Water was subsequently added and the solution was stirred until homogeneous.

TABLE 8

| | BW | BGW | GW |
|---|---|---|---|
| Component | (wt %) | (wt %) | (wt %) |
| 2,3-butanediol¹⁾ | — | 17.5 | 35 |
| 1,3-butanediol²⁾ | 35 | 17.5 | — |
| Phenylethyl alcohol | 10 | 10 | 10 |
| Water | 55 | 55 | 55 |
| | 100 | 100 | 100 |

¹⁾2,3-butanediol; origin: GC CALTEX
²⁾1,3-butylene glycol; origin: Genomatica

The disclosed subject matter has been described with reference to specific details of particular embodiments thereof. It is not intended that such details be regarded as limitations upon the scope of the disclosed subject matter except insofar as and to the extent that they are included in the accompanying claims.

Therefore, the exemplary embodiments described herein are well adapted to attain the ends and advantages mentioned as well as those that are inherent therein. The particular embodiments disclosed above are illustrative only, as the exemplary embodiments described herein may be modified and practiced in different but equivalent manners apparent to those of ordinary skill in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the exemplary embodiments described herein. The exemplary embodiments described herein illustratively disclosed herein suitably may be practiced in the absence of any element that is not specifically disclosed herein and/or any optional element disclosed herein.

1. A fragrance composition comprising at least:
a) 2,3-butanediol and/or 1,3-butanediol, and
b) a fragrance component,
wherein the fragrance composition is free of ethanol and is a homogeneous and transparent solution.

2. The fragrance composition according to claim 1, wherein the fragrance composition comprises 2,3-butanediol and no 1,3-butanediol; comprises 1,3-butanediol and no 2,3-butanediol; or comprises both 2,3-butanediol and 1,3-butanediol.

3. The fragrance composition according to claim 1, wherein 2,3-butanediol is present in an amount from 1% to 95%, by weight relative to the total weight of the fragrance composition.

4. The fragrance composition according to claim 1, wherein 1,3-butanediol is present in an amount from 1% to 95%, by weight relative to the total weight of the fragrance composition.

5. The fragrance composition according to claim 1, wherein the fragrance component is present in an amount from 1% to 99%, by weight relative to the total weight of the fragrance composition.

6. (canceled)

7. (canceled)

8. The fragrance composition according to claim 1, wherein the fragrance component has an average C log P greater than 4.

9. The fragrance composition according to claim 1, wherein the fragrance component comprises at least 20% by weight of ingredients having C log P between 4 and 5, and at least 20% by weight of ingredients having C log P between 5 and 6, relative to the weight of the fragrance component.

10. The fragrance composition according to claim 1, wherein the fragrance composition is free of solvents selected from the group consisting of glycerol, isobutyric acid 1-hydroxy-2,2,4-trimethyl-3-pentyl ester, isobutyric acid-3-hydroxy-2,2,4-trimethyl-1-pentyl ester, and combinations thereof.

11. The fragrance composition according to claim 1, wherein the fragrance composition is free of emulsions and/or surfactants.

12. The fragrance composition according to claim 1, wherein the fragrance composition further comprises water.

13. The fragrance composition according to claim 12, wherein water is present in an amount from 0.1% to 99%, by weight relative to the total weight of the fragrance composition.

14. The fragrance composition according to claim 12, wherein 2,3-butanediol is present in an amount from 1% to 90%, by weight relative to the total weight of the fragrance composition.

15. The fragrance composition according to claim 12, wherein 1,3-butanediol is present in an amount from 1% to 90%, by weight relative to the total weight of the fragrance composition.

16. The fragrance composition according to claim 12, wherein the fragrance component is present in an amount from 1% to 50%, by weight relative to the total weight of the fragrance composition.

17. The fragrance composition according to claim 1, further comprising one or more solvents characterized by a log P greater than or equal to −2 and less than or equal to 2.

18. The fragrance composition according to claim 17, wherein the one or more solvents characterized by a log P greater than or equal to −2 and less than or equal to 2 is selected from the group consisting of glycols, polyalkylene glycols, C₁-C₁₀ alkyl esters of citric acid, and C₃-C₁₀ alkanols.

19. A consumer product comprising the fragrance composition according to claim 1.

20. The consumer product according to claim 19, wherein the consumer product is a perfume, a body-care product, a cosmetic preparation, a skin-care product, a fabric care product, an air care product, or a home care product.

21. The consumer product according to claim 19, wherein the consumer product is a fine perfume, a splash, eau de toilette, eau de parfum, a cologne, a body mist, a body spray, a hair mist, a shave or after-shave lotion, a shampoo, a coloring preparation, a color care product, a hair shaping product, a dental care product, a disinfectant, an intimate care product, a hair spray, a vanishing cream, a deodorant or antiperspirant, hair remover, tanning or sun product, a nail product, a skin cleansing, a makeup, a perfumed soap, a shower or bath mousse, oil or gel, a foot/hand care products, a hygiene product, a liquid or solid or unit-dose detergent, a fabric softener, a solid or liquid fabric scent-booster, a fabric refresher, an ironing water, an air freshener, a “ready to use” powdered air freshener, a mold remover, a furniture care product, a wipe, a dish detergent or hard-surface detergent, a leather care product, or a car care product.

22. A method for enhancing or modulating the perceived olfactive impact and/or long lastingness of a fragrance composition, the method comprising adding 2,3-butanediol and/or 1,3-butanediol to a fragrance component, in the absence of ethanol, to obtain the fragrance composition as a homogeneous and transparent solution.

23. (canceled)
